(19) United States
(12) Patent Application Publication
(10) Pub. No.: US 2015/0320343 A1
(43) Pub. Date: Nov. 12, 2015

(54) MOTION INFORMATION PROCESSING APPARATUS AND METHOD

(71) Applicants: Kabushiki Kaisha Toshiba, Minato-ku (JP); Toshiba Medical Systems Corporation, Otawara-shi (JP)

(72) Inventors: Kazuki UTSUNOMIYA, Nasushiobara (JP); Kousuke SAKAUE, Nasushiobara (JP); Satoshi IKEDA, Yaita (JP)

(73) Assignees: Kabushiki Kaisha Toshiba, Minato-ku (JP); Toshiba Medical Systems Corporation, Otawara-shi (JP)

(21) Appl. No.: 14/802,285

(22) Filed: Jul. 17, 2015

Related U.S. Application Data
(63) Continuation of application No. PCT/JP2014/051015, filed on Jan. 20, 2014.

(30) Foreign Application Priority Data
Jan. 18, 2013 (JP) 2013-007877

(51) Int. Cl. A61B 5/11; A61B 5/00

(52) U.S. Cl. CPC A61B 5/1128; A61B 5/1121; A61B 5/1114; A61B 5/112; A61B 5/743; A61B 5/4824; A61B 5/486; A61B 2505/09

(57) ABSTRACT

A motion information processing apparatus according to an embodiment includes obtaining circuitry, detecting circuitry, and calculating circuitry. The obtaining circuitry obtains depth image information containing coordinate information and depth information of a subject present in a three-dimensional space. The detecting circuitry detects a part of the subject from the depth image information on the basis of the depth information. The calculating circuitry calculates angle information indicating motion in the rotating direction of the part detected from the depth image information by using the coordinate information of the part.

### CROSS-REFERENCE TO RELATED APPLICATIONS

This application is a continuation of PCT international application Ser. No. PCT/JP2014/051015 filed on Jan. 20, 2014 which designates the United States, incorporated herein by reference, and which claims the benefit of priority from Japanese Patent Application No. 2013-007877, filed on Jan. 18, 2013, the entire contents of which are incorporated herein by reference.

### FIELD

Embodiments described herein relate generally to a motion information processing apparatus and a method therefor.

### BACKGROUND

In rehabilitation, support has been provided by many experts working in cooperation for the purpose of helping those experiencing mental or physical disabilities due to various causes such as illnesses, injuries, or aging or those having congenital disorders to lead better lives. For example, rehabilitation involves support provided by many experts such as rehabilitation specialists, rehabilitation nurses, physical therapists, occupational therapists, speech-language-hearing therapists, clinical psychologists, prosthetists and orthoptists, and social workers working in cooperation.

In the meantime, in recent years, development of motion capture technologies for digitally recording motions of people and objects has been advancing. Examples of systems of the motion capture technologies that are known include optical, mechanical, magnetic, and camera systems. For example, a camera system of digitally recording motions of a person by making the person wear a marker, detecting the marker by a tracker such as a camera, and processing the detected marker is known. For another example, as a system that does not use markers and trackers, a system of digitally recording motions of a person by using an infrared sensor to measure the distance from the sensor to the person and detect the size and various motions of the skeleton of the person is known. Kinect (registered trademark), for example, is known as a sensor using such a system.

### BRIEF DESCRIPTION OF THE DRAWINGS

FIG. 1 is a block diagram illustrating an example configuration of a motion information processing apparatus according to a first embodiment;

FIG. 2A is a diagram for explaining processing of motion information generating circuitry according to the first embodiment;

FIG. 2B is a diagram for explaining processing of the motion information generating circuitry according to the first embodiment;

FIG. 2C is a diagram for explaining processing of the motion information generating circuitry according to the first embodiment;

FIG. 3 is a table illustrating an example of skeleton information generated by the motion information generating circuitry according to the first embodiment;

FIG. 4 is a diagram for explaining rotating motion of a forearm;

FIG. 5 is a block diagram illustrating a detailed example configuration of the motion information processing apparatus according to the first embodiment;

FIG. 6A is a diagram for explaining processing performed by setting circuitry according to the first embodiment;

FIG. 6B is a diagram for explaining processing performed by the setting circuitry according to the first embodiment;

FIG. 7 is a diagram for explaining processing performed by detecting circuitry according to the first embodiment;

FIG. 8 is a diagram for explaining processing performed by calculating circuitry according to the first embodiment;

FIG. 9 is a diagram for explaining processing performed by display controlling circuitry according to the first embodiment;

FIG. 10 is a flowchart for explaining an example of procedures of a calculation process according to the first embodiment;

FIG. 11 is a flowchart for explaining an example of procedures of a process for displaying a display image according to the first embodiment;

FIG. 12 is a flowchart for explaining an example of procedures of a process for displaying a graph according to the first embodiment;

FIG. 13 is a flowchart for explaining an example of procedures of a process for displaying a maximum rotation angle according to the first embodiment;

FIG. 14 is a diagram for explaining processing performed by detecting circuitry according to a second embodiment;

FIG. 15 is a flowchart for explaining an example of procedures of an angle calculation process according to the second embodiment; and

FIG. 16 is a diagram for explaining an example of application to a service providing apparatus.

### DETAILED DESCRIPTION

A motion information processing apparatus according to an embodiment includes obtaining circuitry, detecting circuitry, and calculating circuitry. The obtaining circuitry obtains depth image information containing coordinate information and depth information of a subject present in a three-dimensional space. The detecting circuitry detects a part of the subject from the depth image information on the basis of the depth information. The calculating circuitry calculates angle information indicating motion in the rotating direction of the part detected from the depth image information by using the coordinate information of the part.

Hereinafter, motion information processing apparatuses and programs therefor according to embodiments will be described with reference to the drawings. Note that the motion information processing apparatuses described below may be used alone or may be embedded in a system such as a medical record system or a rehabilitation department system, for example.

### First Embodiment

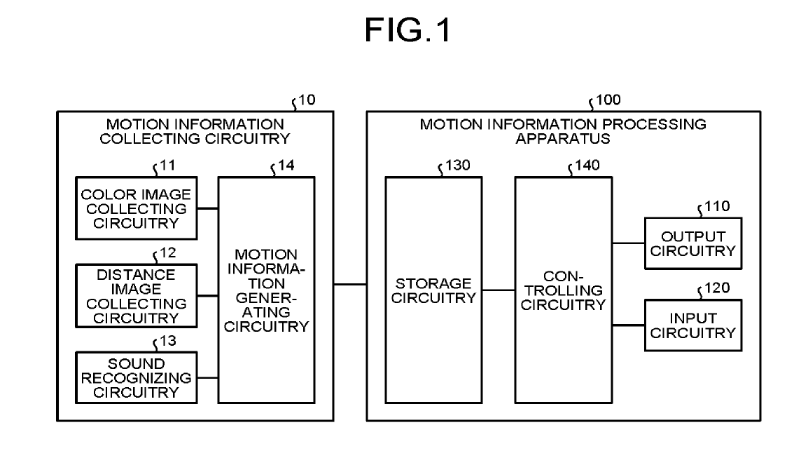

FIG. 1 is a block diagram illustrating an example configuration of a motion information processing apparatus 100 according to a first embodiment. The motion information processing apparatus 100 according to the first embodiment is a apparatus to support rehabilitation in a medical institution, at home, in an office, or the like. Note that “rehabilitation” refers to techniques and methods for developing the potentials of patients with disabilities, chronic diseases, geriatric diseases and the like receiving prolonged treatment, and restoring and promoting their vital functions and also their social functions. Examples of such techniques and methods include functional exercises for restoring and promoting vital functions and social functions. Note that examples of the functional exercises include gait training and range of motion exercise. A person who undergoes rehabilitation will be referred to as a “subject.” Examples of the subject include a sick person, an injured person, an aged person, and a handicapped person. In addition, a person who assists a subject in rehabilitation will be referred to as a “caregiver.” Examples of the caregiver include healthcare professionals such as a doctor, a physical therapist, and a nurse working at medical institutions, and a care worker, a family member, and a friend caring a subject at home, for example. Furthermore, rehabilitation will also be abbreviated as “rehab.”

As illustrated in FIG. 1, in the first embodiment, the motion information processing apparatus 100 is connected to a motion information collecting circuitry 10.

The motion information collecting circuitry 10 detects motion of a person, an object, or the like in a space in which rehabilitation is carried out, and collects motion information representing the motion of the person, the object, or the like. The motion information will be described in detail later in the description of processing performed by motion information generating circuitry 14. For the motion information collecting circuitry 10, Kinect (registered trademark) is used, for example.

As illustrated in FIG. 1, the motion information collecting circuitry 10 includes color image collecting circuitry 11, distance image collecting circuitry 12, sound recognizing circuitry 13, and the motion information generating circuitry 14. Note that the configuration of the motion information collecting circuitry 10 illustrated in FIG. 1 is only an example, and the embodiment is not limited thereto.

The color image collecting circuitry 11 photographs a subject such as a person, an object, or the like in a space in which rehabilitation is carried out, and collects color image information. The color image collecting circuitry 11 detects light reflected by a surface of the subject by a photodetector, and converts visible light into an electrical signal, for example. The color image collecting circuitry 11 then generates one frame of color image information corresponding to the photographed range by converting the electrical signal into digital data. The color image information of one frame contains photographing time information, and information of pixels contained in the frame and RGB (red, green, and blue) values with which the respective pixels are associated, for example. The color image collecting circuitry 11 takes a moving image of the photographed range by generating multiple successive frames of color image information from visible light detected successively. Note that the color image information generated by the color image collecting circuitry 11 may be output as a color image in which the RGB values of the pixels are arranged in a bitmap. The color image collecting circuitry 11 has a complementary metal oxide semiconductor (CMOS) or a charge coupled device (CCD), for example, as the photodetector.

The distance image collecting circuitry 12 photographs a subject such as a person, an object, or the like in a space in which rehabilitation is carried out, and collects distance image information. The distance image collecting circuitry 12 irradiates a surrounding area with infrared light and detects with a photodetector a reflected wave that is the irradiation wave reflected by a surface of the subject, for example. The distance image collecting circuitry 12 then obtains the distance between the subject and the distance image collecting circuitry 12 on the basis of a phase difference between the irradiation wave and the reflected wave and on the time from the irradiation to the detection, and generates one frame of distance image information corresponding to the photographed range. The distance image information of one frame contains photographing time information, and information of pixels contained in the photographed range and the distances between the subject and the distance image collecting circuitry 12 with which the respective pixels are associated, for example. The distance image collecting circuitry 12 takes a moving image of the photographed range by generating multiple successive frames of distance image information from reflected waves detected successively. Note that the distance image information generated by the distance image collecting circuitry 12 may be output as a distance image in which shades of colors according to the distances of the pixels are arranged in a bitmap. The distance image collecting circuitry 12 has a CMOS or a CCD, for example, as the photodetector. The photodetector may also be used in common as the photodetector used in in the color image collecting circuitry 11. The unit of a distance calculated by the distance image collecting circuitry 12 is meter [m], for example.

The sound recognizing circuitry 13 collects sound there around, and carries out determination of the direction of a sound source and sound recognition. The sound recognizing circuitry 13 has a microphone array including multiple microphone, and carries out beamforming. Beamforming is a technique for selectively collecting sound from a particular direction. The sound recognizing circuitry 13 determines the direction of a sound source through beamforming using the microphone array, for example. The sound recognizing circuitry 13 also recognizes words from collected sound by using a known sound recognition technology. Specifically, the sound recognizing circuitry 13 generates information of a word recognized according to the sound recognition technology with which the direction from which the word has been uttered and the time when the word has been recognized are associated, for example, as a sound recognition result.

The motion information generating circuitry 14 generates motion information indicating a motion of a person, an object, or the like. The motion information is generated by regarding a motion (gesture) of a person as a series of multiple postures (poses), for example. The outline will be explained as follows. The motion information generating circuitry 14 first obtains coordinates of joints forming a human body skeleton from the distance image information generated by the distance image collecting circuitry 12 by pattern matching using human body patterns. The coordinates of the joints obtained from the distance image information are values expressed in a coordinate system of a distance image (hereinafter referred to as a “distance image coordinate system”). Thus, the motion information generating circuitry 14 then converts the coordinates of the joints in the distance image coordinate system into values expressed in a coordinate system of a three-dimensional space in which rehabilitation is carried out (hereinafter referred to as a “world coordinate system”). The coordinates of the joint expressed in the world coordinate system constitute skeleton information of one frame. Furthermore, skeleton information of multiple frames constitutes motion information. Hereinafter, processing performed by the motion information generating circuitry 14 according to the first embodiment will be described more concretely.

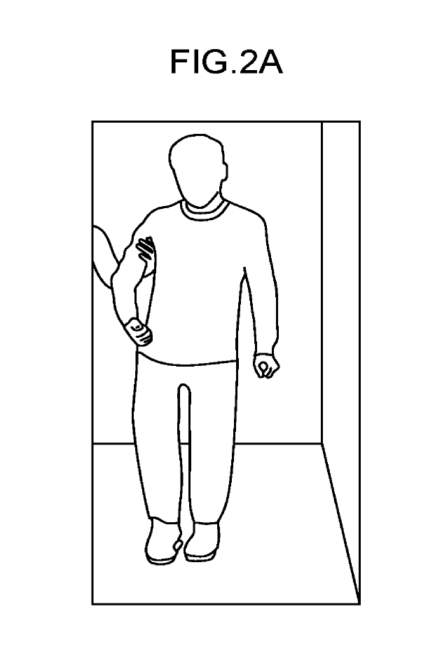

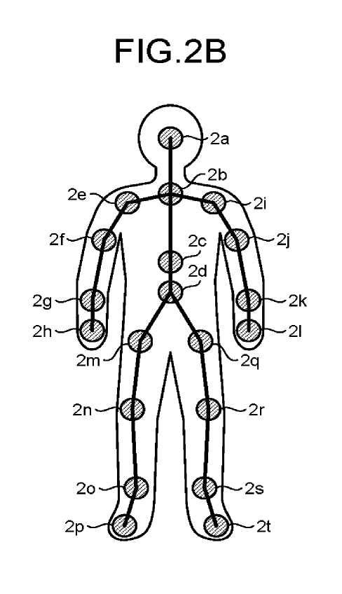

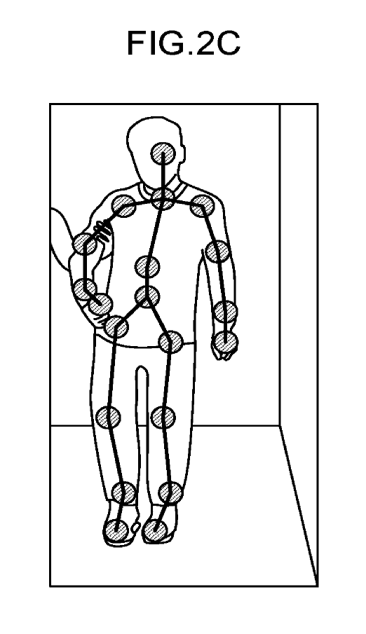

FIGS. 2A to 2C are diagrams for explaining processing performed by the motion information generating circuitry 14 according to the first embodiment. FIG. 2A illustrates an example of a distance image taken by the distance image collecting circuitry 12. Note that, in FIG. 2A, an image expressed by line drawing is presented for the purpose of illustration, an actual distance image is an image expressed by color shadings according to the distances, or the like. In this distance image, each pixel has three-dimensional values, which are a “pixel position X” in the horizontal direction of the distance image, a “pixel position Y” in the vertical direction of the distance image, and a “distance Z” between the subject corresponding to the pixel and the distance image collecting circuitry 12. Hereinafter, coordinate values in the distance image coordinate system will be expressed by the three-dimensional values (X, Y, Z).

In the first embodiment, the motion information generating circuitry 14 stores human body patterns corresponding to various postures through learning in advance. Each time distance image information is generated by the distance image collecting circuitry 12, the motion information generating circuitry 14 acquires the generated distance image information of each frame. The motion information generating circuitry 14 then carries out pattern matching on the acquired distance image information of each frame using the human patterns.

Here, the human patterns will be described. FIG. 2B illustrates an example of the human patterns. In the first embodiment, the human patterns are patterns used in pattern matching with the distance image information, and are thus expressed in the distance image coordinate system and have information on the surfaces of human bodies (hereinafter referred to as “human body surfaces”) similarly to a person drawn in a distance image. A human body surface corresponds to the skin or the surface of clothing of the person, for example. Furthermore, a human body pattern has information on joints forming human skeleton as illustrated in FIG. 2B. Thus, in a human pattern, relative positions of a human body surface and the joints are known.

In the example illustrated in FIG. 2B, the human body pattern has information on 20 joints, from a joint 2a to a joint 2t. The joint 2a corresponds to the head, the joint 2b corresponds to the center of the shoulders, the joint 2c corresponds to the waist, and the joint 2d corresponds to the center of the hip. The joint 2e corresponds to the right shoulder, the joint 2f corresponds to the right elbow, the joint 2g corresponds to the right wrist, and the joint 2h corresponds to the right hand. The joint 2i corresponds to the left shoulder, the joint 2j corresponds to the left elbow, the joint 2k corresponds to the left wrist, and the joint 2l corresponds to the left hand. The joint 2m corresponds to the right hip, the joint 2n corresponds to the right knee, the joint 20 corresponds to the right ankle, and the joint 2p corresponds to the tarsus of the right foot. The joint 2q corresponds to the left hip, the joint 2r corresponds to the left knee, the joint 2s corresponds to the left ankle, and the joint 2t corresponds to the tarsus of the left foot.

While a case in which the human body pattern has information on 20 joints is illustrated in FIG. 2B, the embodiment is not limited thereto, and the positions and the number of joints may be arbitrarily be set by an operator. For example, for capturing only a change in the motion of the limbs, information on the joint 2b and the joint 2c of the joints 2a to 2d need not be acquired. For capturing a change in the motion of the right hand in detail, joints of the fingers of the right hand may be newly set in addition to the joint 2h. Note that, although the joint 2a, the joint 2h, the joint 2l, the joint 2p, and the joint 2t in FIG. 2B are at distal portions of bones and are thus different from what are actually called joints, these points will be referred to as joints for the purpose of explanation since the points are important points for indicating the positions and orientations of the bones.

The motion information generating circuitry 14 carries out pattern matching with the distance image information of each frame by using such human body patterns. For example, the motion information generating circuitry 14 carries out pattern matching between the human body surface of the human body pattern illustrated in FIG. 2B and the distance image illustrated in FIG. 2A to extract a person in a certain posture from the distance image information. In this manner, the motion information generating circuitry 14 obtains the coordinates of the human body surface of the person drawn in the distance image. Furthermore, as described above, in a human pattern, relative positions of a human body surface and joints are known. The motion information generating circuitry 14 thus calculates the coordinates of the joints in the person drawn in the distance image from the coordinates of the human body surface of the person. In this manner, as illustrated in FIG. 2C, the motion information generating circuitry 14 obtains the coordinates of the joints forming the human body skeleton from the distance image information. Note that the coordinates of the joints obtained here are coordinates in the distance image coordinate system.

Note that the motion information generating circuitry 14 may use information indicating relative positions of the joints supplementarily in carrying out the pattern matching. The information indicating the relative positions of the joints contains connections between joints (“connection between the joint 2a and the joint 2b,” for example), and the ranges of motion of the joints, for example. A joint is a part connecting two or more bones. The angle between bones changes with a change in posture, and the ranges of range are different for different joints. A range of motion is expressed by the largest value and the smallest value of the angle between bones that the joint connects, for example. In learning a human body pattern, the motion information generating circuitry 14 also learns the ranges of motion of the joints and stores the learned ranges of motion in association with the respective joints, for example.

Subsequently, the motion information generating circuitry 14 converts the coordinates of the joints in the distance image coordinate system into values expressed in the world coordinate system. The world coordinate system refers to a coordinate system of a three-dimensional space in which rehabilitation is carried out, such as a coordinate system with the origin at the position of the motion information collecting circuitry 10, the x-axis in the horizontal direction, the y-axis in the vertical direction, and the z-axis in a direction perpendicular to the xy plane. Note that a coordinate value in the z-axis direction may be referred to as a “depth.”

Here, processing of conversion from the distance image coordinate system to the world coordinate system will be described. In the first embodiment, it is assumed that the motion information generating circuitry 14 stores in advance a conversion formula for conversion from the distance image coordinate system to the world coordinate system. Coordinates in the distance image coordinate system and an entrance angle of reflected light associated with the coordinates are input to this conversion formula and coordinates in the world coordinate system are output therefrom, for example. The motion information generating circuitry 14 inputs coordinates (X1, Y1, Z1) of a joint and the entrance angle of reflected light associated with the coordinates to the conversion formula, and converts the coordinates (X1, Y1, Z1) of the joint into coordinates (x1, y1, z1) of the world coordinate system, for example. Note that, since the relation between the coordinates in the distance image coordinate system and the entrance angle of reflected light is known, the motion information generating circuitry 14 can input the entrance angle associated with the coordinates (X1, Y1, Z1) into the conversion formula. Although a case in which the motion information generating circuitry 14 converts coordinates in the distance image coordinate system into coordinates in the world coordinate system has been described here, the motion information generating circuitry 14 may alternatively convert coordinates in the world coordinate system into coordinates in the distance image coordinate system.

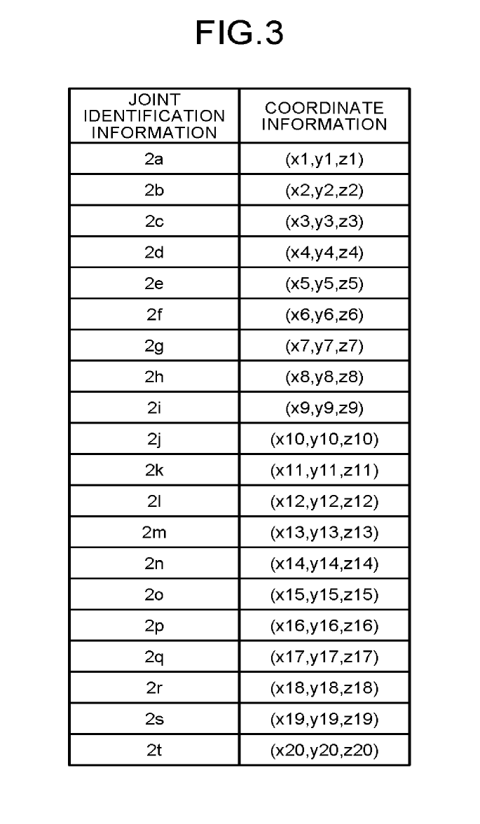

The motion information generating circuitry 14 then generates skeleton information from the coordinates of the joints expressed in the world coordinate system. FIG. 3 is a table illustrating an example of the skeleton information generated by the motion information generating circuitry 14. The skeleton information of each frame contains photographing time information of the frame and the coordinates of the joints. The motion information generating circuitry 14 generates skeleton information containing joint identification information and coordinate information associated with each other as illustrated in FIG. 3, for example. Note that the photographing time information is not illustrated in FIG. 3. The joint identification information is identification information for identifying a joint, and is set in advance. For example, joint identification information “2a” corresponds to the head, and joint identification information “2b” corresponds to the center of the shoulders. The other joint identification information data similarly indicate the respective corresponding joints. The coordinate information indicates coordinates of each joint in each frame in the world coordinate system.

In the first row of FIG. 3, the joint identification information “2a” and the coordinate information “(x1, y1, z1)” are associated. Specifically, the skeleton information of FIG. 3 indicates that the head is present at the position of coordinates (x1, y1, z1) in a certain frame. In addition, in the second row of FIG. 3, the joint identification information “2b” and the coordinate information “(x2, y2, z2)” are associated. Specifically, the skeleton information of FIG. 3 indicates that the center of the shoulders is present at the position of coordinates (x2, y2, z2) in a certain frame. Similarly for the other joints, the skeleton information indicates that each joint is present at a position expressed by the corresponding coordinates in a certain frame.

In this manner, the motion information generating circuitry 14 carries out pattern matching on the distance image information of each frame each time the distance image information of each frame is acquired from the distance image collecting circuitry 12, and converts the coordinates from the distance image coordinate system into those in the world coordinate system to generate the skeleton information of each frame. The motion information generating circuitry 14 then outputs the generated skeleton information of each frame to the motion information processing apparatus 100 to store the skeleton information in motion information storage circuitry 131, which will be described later.

Note that the processing of the motion information generating circuitry 14 is not limited to the technique described above. For example, although a technique in which the motion information generating circuitry 14 carries out pattern matching using human body patterns has been described above, the embodiment is not limited thereto. For example, a technique in which patterns of each part is used instead of or in addition to the human body patterns may be used.

Furthermore, for example, although a technique in which the motion information generating circuitry 14 obtains coordinates of joints from the distance image information has been described above, the embodiment is not limited thereto. For example, a technique in which the motion information generating circuitry 14 obtains coordinates of joints by using color image information in addition to the distance image information may be used. In this case, the motion information generating circuitry 14 carries out pattern matching between a human body pattern expressed in a coordinate system of a color image and the color image information, and obtains coordinates of the human body surface from the color image information, for example. The coordinate system of the color image does not include information corresponding to the “distance Z” in the distance image coordinate system. Thus, the motion information generating circuitry 14 obtains the information on the “distance Z” from the distance image information, for example, and obtains coordinates of joints in the world coordinate system through a calculation process using these two information data.

The motion information generating circuitry 14 also outputs color image information generated by the color image collecting circuitry 11, distance image information generated by the distance image collecting circuitry 12, and a sound recognition result output from the sound recognizing circuitry 13, where necessary, to the motion information processing apparatus 100 to store the color image information, the distance image information, and the sound recognition result in the motion information storage circuitry 131, which will be described later. Note that a pixel position in the color image information and a pixel position in the distance image information can be associated with each other in advance according to the positions of the color image collecting circuitry 11 and the distance image collecting circuitry 12 and the photographing direction. Thus, a pixel position in the color image information and a pixel position in the distance image information can also be associated with the world coordinate system calculated by the motion information generating circuitry 14. Furthermore, the height and the lengths of body parts (the length of an arm, the length of the abdomen, etc.) can be obtained or the distance between two pixels specified on a color image can be obtained by using the association and a distance [m] calculated by the distance image collecting circuitry 12. Similarly, the photographing time information in the color image information and the photographing time information in the distance image information can also be associated with each other in advance. In addition, the motion information generating circuitry 14 can refer to the sound recognition result and the distance image information, and if a joint 2a is present about the direction in which a word recognized through sound recognition at certain time has been uttered, can output the word as a word uttered by a person having the joint 2a. Furthermore, the motion information generating circuitry 14 also outputs information indicating relative positions of the joints, where necessary, to the motion information processing apparatus 100 to store the information in the motion information storage circuitry 131, which will be described later.

The motion information generating circuitry 14 also generates depth image information of one frame corresponding to the photographed range by using a depth that is a coordinate value in the z-axis direction of the world coordinate system. The depth image information of one frame contains photographing time information, and information of pixels contained in the photographed range with which the depths associated with the respective pixels are associated, for example. In other words, the depth image information associates the pixels with depth information instead of the distance information with which the pixels in the distance image information are associated, and can indicate the pixel positions in the distance image coordinate system similar to that of the distance image information. The motion information generating circuitry 14 outputs the generated depth image information to the motion information processing apparatus 100 to store the depth image information in depth image information storage circuitry 132, which will be described later. Note that the depth image information may be output as a depth image in which shades of colors according to the depths of the pixels are arranged in a bitmap.

Although a case in which motion of one person is detected by the motion information collecting circuitry 10 has been described here, the embodiment is not limited thereto. If multiple people are included in the photographed range of the motion information collecting circuitry 10, the motion information collecting circuitry 10 may detect motions of multiple people. If multiple people are photographed in distance image information of the same frame, the motion information collecting circuitry 10 associates the skeleton information data of the multiple people generated from the distance image information of the same frame, and outputs the associated skeleton information data as motion information to the motion information processing apparatus 100.

Note that the configuration of the motion information collecting circuitry 10 is not limited to the configuration described above. For example, in a case where motion information is generated by detecting motion of a person through another motion capture technology such as an optical, mechanical, or magnetic technology, the motion information collecting circuitry 10 need not necessarily include the distance image collecting circuitry 12. In such a case, the motion information collecting circuitry 10 includes a marker to be worn by a human body to detect the motion of a person and a sensor for detecting the marker as a motion sensor. The motion information collecting circuitry 10 then detects the motion of the person by using the motion sensor and generates motion information. The motion information collecting circuitry 10 also associates pixel positions of the color image information and coordinates of the motion information with each other by using the positions of the marker contained in the image photographed by the color image collecting circuitry 11, and outputs the association result to the motion information processing apparatus 100 where necessary. In addition, for example, if the motion information collecting circuitry 10 does not output the sound recognition result to the motion information processing apparatus 100, the motion information collecting circuitry 10 need not have the sound recognizing circuitry 13.

Furthermore, although the motion information collecting circuitry 10 outputs coordinates in the world coordinate system as the skeleton information in the embodiment described above, the embodiment is not limited thereto. For example, the motion information collecting circuitry 10 may output coordinates in the distance image coordinate system before conversion, and the conversion from the distance image coordinate system to the world coordinate system may be carried out in the motion information processing apparatus 100 where necessary.

The description refers back to FIG. 1. The motion information processing apparatus 100 performs processing for supporting rehabilitation by using the motion information output from the motion information collecting circuitry 10. The motion information processing apparatus 100 is an information processing apparatus such as a computer or a workstation, for example, and includes output circuitry 110, input circuitry 120, storage circuitry 130, and controlling circuitry 140 as illustrated in FIG. 1.

The output circuitry 110 outputs various information data for supporting rehabilitation. For example, the output circuitry 110 displays a graphical user interface (GUI) for an operator who operates the motion information processing apparatus 100 to input various request by using the input circuitry 120, displays an output image and the like generated by the motion information processing apparatus 100, or outputs an alarm. The output circuitry 110 is a monitor, a speaker, a headphone, or a headphone part of a headset, for example. The output circuitry 110 may be a display that is worn on the body of a user such as a spectacle type display or a head mounted display.

The input circuitry 120 receives input of various information data for supporting rehabilitation. For example, the input circuitry 120 receives input of various requests from the operator of the motion information processing apparatus 100, and transfers the received requests to the motion information processing apparatus 100. The input circuitry 120 is a mouse, a keyboard, a touch command screen, a trackball, a microphone, or a microphone part of a headset, for example. The input circuitry 120 may be a sensor for acquiring biological information such as a sphygmomanometer, a heart rate monitor, or a clinical thermometer.

The storage circuitry 130 is a storage device such as a semiconductor memory device such as a random access memory (RAM) and a flash memory, a hard disk device, or an optical disk device, for example. The controlling circuitry 140 can be an integrated circuit such as an application specific integrated circuit (ASIC) or a field programmable gate array (FPGA), or can be implemented in a predetermined program executed by a central processing unit (CPU).

The configuration of the motion information processing apparatus 100 according to the first embodiment has been described above. With such a configuration, the motion information processing apparatus 100 according to the first embodiment analyzes motion information of a subject carrying out rehab collected by the motion information collecting circuitry 10 to support the rehab of the subject.

Note that the motion information processing apparatus 100 according to the first embodiment can evaluate motion in a rotating direction through a process described below. The motion information processing apparatus 100 can evaluate rotating motion of a forearm that is difficult to evaluate only on the basis of coordinates of joints, for example.

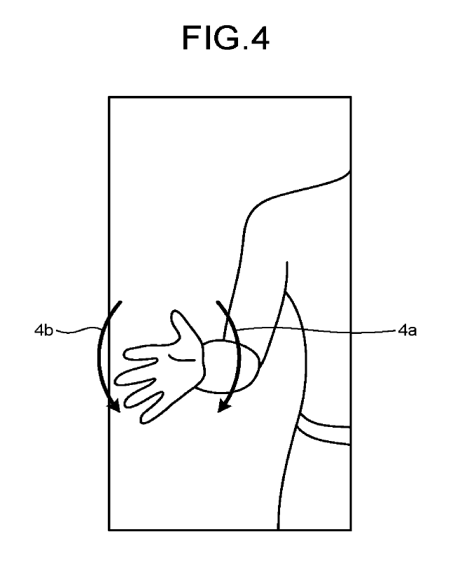

FIG. 4 is a diagram for explaining rotating motion of a forearm. The rotating motion of a forearm includes two motions, which are pronation and supination. FIG. 4 illustrates a case in which a person performs rotating motion of the right arm. In the example illustrated in FIG. 4, the person holds his/her right forearm (a part from the right elbow to the right wrist) horizontally, the palm of the right hand facing the observer's right and the back of the right hand facing the observer's left. In this state, without changing the position of the forearm, rotation in a direction 4a in which the right palm turns down is referred to as pronation and rotation in a direction 4b in which the right palm turns up is referred to as supination.

Note that the rotating motion is difficult to evaluate by applying the motion information described above to the person in FIG. 4 and acquiring coordinates of the joint 2f (right elbow) and the joint 2g (right wrist) related to the right forearm. Specifically, when pronation and supination of the right arm is performed, the coordinates of the joint 2f and the joint 2g do not change, which is why it is difficult to evaluate rotating motion. Thus, the motion information processing apparatus 100 according to the first embodiment enables evaluation of motion in a rotating direction through a process described below.

In the following, a case in which the motion information processing apparatus 100 evaluates rotating motion of a forearm will be described, but the embodiment is not limited thereto. For example, the motion information processing apparatus 100 can also be applied to evaluation of rotating motion of a shoulder and a hip joint, and further to motion in the rotating direction that can be evaluated only on the basis of coordinates of joints. Thus, the motion information processing apparatus 100 according to the first embodiment provides a new method for evaluating motion in the rotating direction.

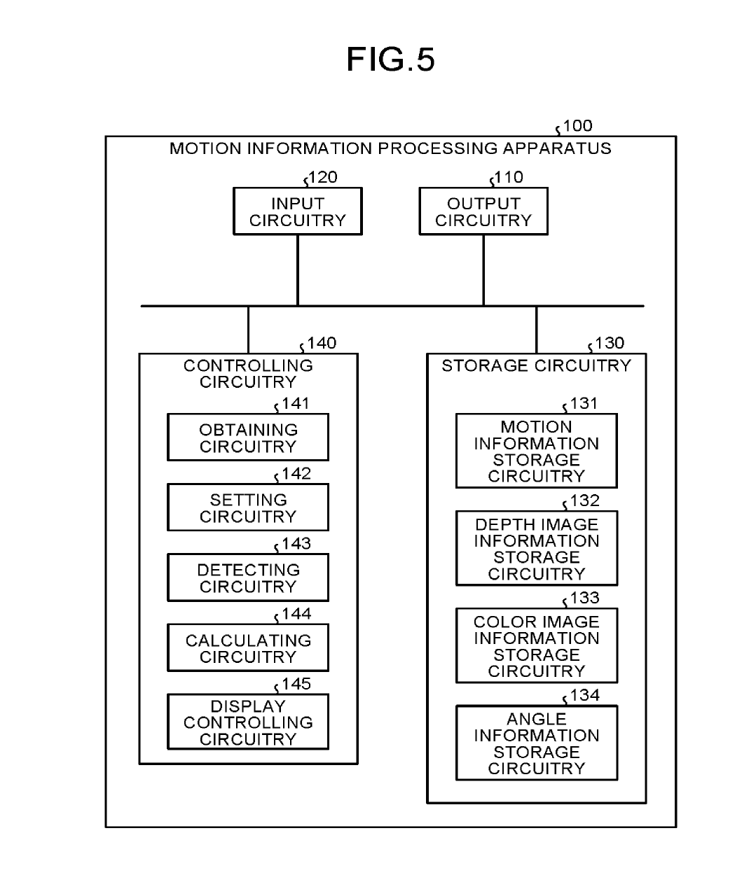

FIG. 5 is a block diagram illustrating a detailed example configuration of the motion information processing apparatus 100 according to the first embodiment. As illustrated in FIG. 5, in the motion information processing apparatus 100, the storage circuitry 130 includes the motion information storage circuitry 131, the depth image information storage circuitry 132, color image information storage circuitry 133, and angle information storage circuitry 134.

The motion information storage circuitry 131 stores motion information data collected by the motion information collecting circuitry 10. The motion information is skeleton information of each frame generated by the motion information generating circuitry 14. The motion information is stored in the motion information storage circuitry 131 each time the motion information is collected by the motion information collecting circuitry 10, for example.

The depth image information storage circuitry 132 stores depth image information generated by the motion information collecting circuitry 10. The depth image information is stored in the depth image information storage circuitry 132 each time the depth image information is generated by the motion information collecting circuitry 10, for example.

The color image information storage circuitry 133 stores color image information collected by the motion information collecting circuitry 10. The color image information is stored in the color image information storage circuitry 133 each time the color image information is collected by the motion information collecting circuitry 10, for example.

Note that, in the motion information storage circuitry 131, the depth image information storage circuitry 132, and the color image information storage circuitry 133, coordinates of joints in the skeleton information, pixel positions in the depth image information, and pixel positions in the color image information are associated with one another in advance. Photographing time information in the skeleton information, photographing time information in the depth image information, and photographing time information in the color image information are also associated with one another in advance.

The angle information storage circuitry 134 stores information indicating an angle of a part to be processed, for example. For evaluation of rotating motion of a left arm, for example, the angle information storage circuitry 134 stores information indicating the angle of the left hand to the horizontal direction of a depth image of each frame. The information to be stored in the angle information storage circuitry 134 is calculated by calculating circuitry 144, which will be described later. Note that the information to be stored in the angle information storage circuitry 134 is not limited thereto. For example, the angle information storage circuitry 134 may store angular velocity that is an amount of change with time of the angle of the left hand to the horizontal direction of a depth image.

In the motion information processing apparatus 100, the controlling circuitry includes obtaining circuitry 141, setting circuitry 142, detecting circuitry 143, the calculating circuitry 144, and display controlling circuitry 145.

The obtaining circuitry 141 obtains depth image information containing coordinate information and depth information of a subject present in a space in which rehabilitation is carried out. For example, each time motion information collecting circuitry 10 and the motion information processing apparatus 100 are powered on and skeleton information of one frame is stored in the motion information storage circuitry 131, the obtaining circuitry 141 obtains the skeleton information, and depth image information and color image information of the corresponding frame from the motion information storage circuitry 131, the depth image information storage circuitry 132, and the color image information storage circuitry 133, respectively.

The setting circuitry 142 sets a detection space containing a part to be processed. For example, the setting circuitry 142 receives an input to specify a part that is a target of rehabilitation and an exercise from a user via the input circuitry 120. Subsequently, the setting circuitry 142 extracts coordinates of the joint 2l to be processed from the motion information obtained by the obtaining circuitry 141 according to the part and exercise specified by the input. The setting circuitry 142 then sets a detection space containing the extracted coordinates of the joint in the space in which rehabilitation is carried out.

Note that the setting circuitry 142 sets the detection space to narrow down the space in which motion in the rotating direction is performed in the space in which rehabilitation is carried out. Specifically, the space in which motion in the rotating direction is carried out is narrowed down in the x, y, and z directions. As a result of narrowing the space down in the x and y directions, the motion in the rotating direction performed by a subject can be distinguished from another motion or a positional change of another object or person and analyzed. In a specific example, in a case where rotating motions of both forearms are performed, the rotating motions of the forearms can also be analyzed by setting detection spaces with the positions of the right hand and the left hand at the centers. Note that the motion in the rotating direction performed in the detection space can be recognized as an image by analyzing an image taken in a photographing direction that is substantially the same as the rotation axis. Details of this process will be described later.

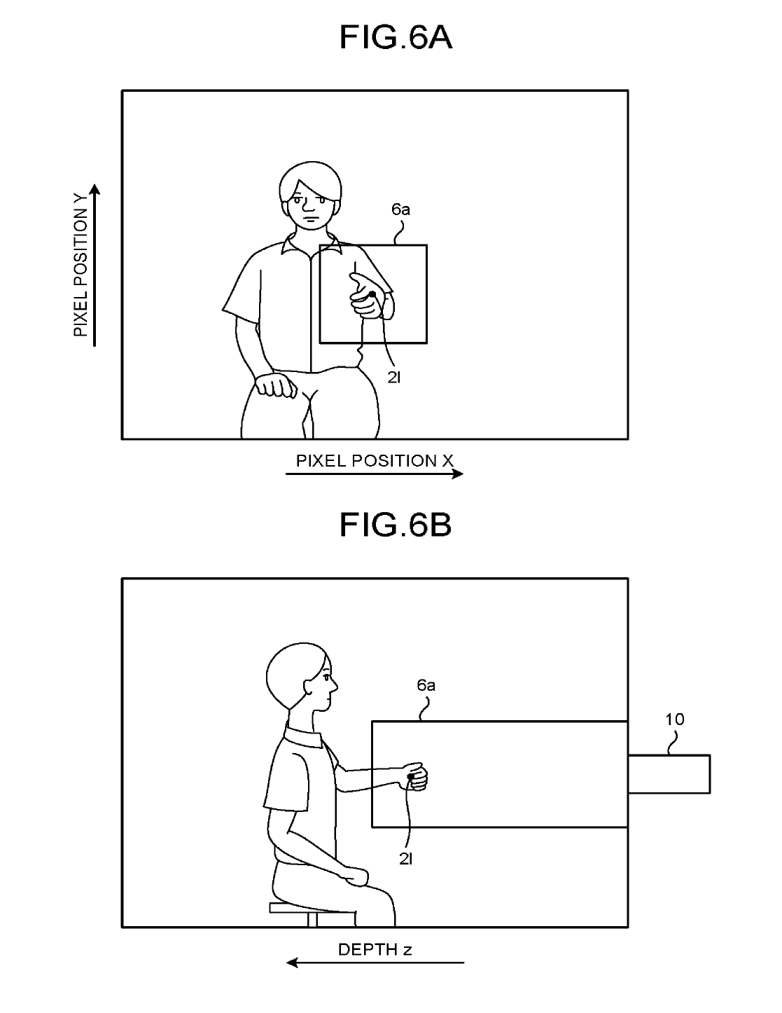

FIGS. 6A and 6B are diagrams for explaining processing performed by the setting circuitry 142 according to the first embodiment. FIGS. 6A and 6B illustrate a case in which a person performs rotating motion of the left forearm. In this case, the setting circuitry 142 is assumed to have received an input indicating that rotation motion of the left forearm will be performed from a user via the input circuitry 120. Note that FIG. 6A is a front view of the person performing the rotating motion, and corresponds to a color image taken by the motion information collecting circuitry 10. The horizontal direction of the color image corresponds to a “pixel position X” in the distance image coordinate system, and the vertical direction of a color image corresponds to a “pixel position Y” in the distance image coordinate system. FIG. 6B is a lateral view of the person performing the rotating motion, and the leftward direction of FIG. 6B corresponds to the z-axis direction in the world coordinate system, that is, the depth.

As illustrated in FIGS. 6A and 6B, upon receiving the input indicating that rotating motion of the left forearm will be performed, the setting circuitry 142 extracts the coordinates of the joint 2l of the left hand from the motion information obtained by the obtaining circuitry 141. The setting circuitry 142 then sets a detection space 6a containing the extracted coordinates of the joint 2l in the space in which rehabilitation is carried out. The detection space 6a is expressed by the world coordinate system. Specifically, for example, the x-axis direction of the detection space 6a is set to a range of 30 cm with the center thereof at the value in the x-axis direction of the joint 2l. The y-axis direction of the detection space 6a is set to a range of 30 cm with the center thereof at the value in the y-axis direction of the joint 2l. Thus, as illustrated in FIG. 6A, the range in the x-axis direction and the range in the y-axis direction of the detection space 6a are expressed in a color image by being converted to the distance image coordinate system (the range of the pixel position X and the range of the pixel position Y, respectively). Furthermore, the z-axis direction of the detection space 6a is set to a range from a position at a value obtained by multiplying the value in the z-axis direction of the joint 2l by 1.2 to the position of the motion information collecting circuitry 10 as illustrated in FIG. 6B. In this manner, the setting circuitry 142 sets a space having a shape of a prism containing the position of the joint to be processed to be the detection space. Note that the detection space set by the setting circuitry 142 is not limited to the example described above, but the values may be changed in any manner depending on the part to be processed. The setting circuitry 142 may alternatively set a space having any shape such as a shape of a regular hexahedron or a spherical shape to be the detection space.

The detecting circuitry 143 detects a part of a subject from the depth image information on the basis of depth information. For example, the detecting circuitry 143 detects the part to be processed by binarizing the depth image information by using the detection space set by the setting circuitry 142.

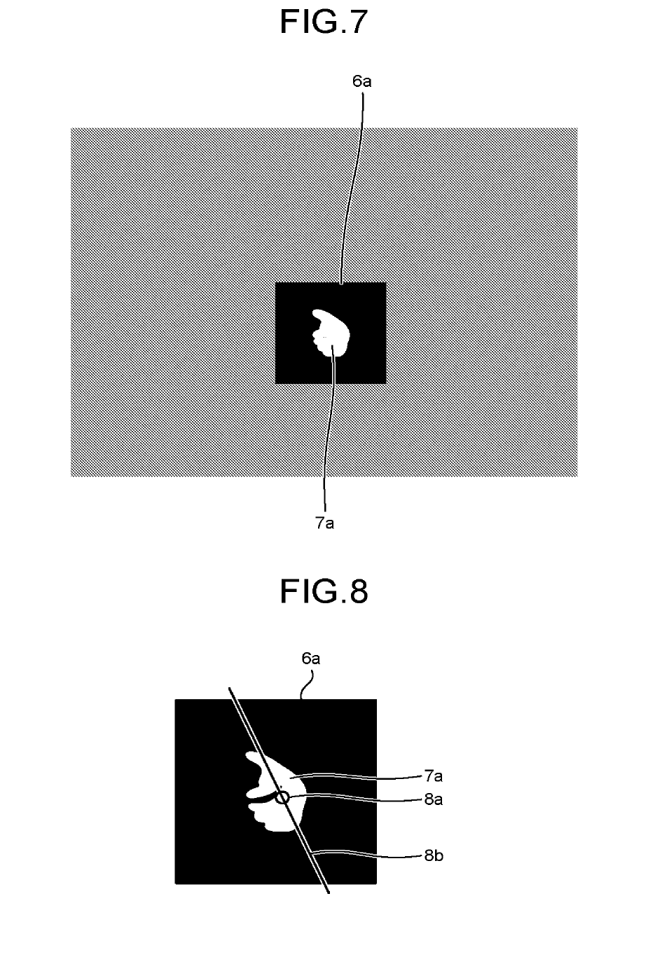

FIG. 7 is a diagram for explaining processing performed by the detecting circuitry 143 according to the first embodiment. FIG. 7 illustrates a case in which a depth image corresponding to that in FIG. 6A is binarized. As illustrated in FIG. 7, the detecting circuitry 143 sets an area surrounded by the range in the x-axis direction and the range in the y-axis direction of the detection space 6a in the depth image obtained by the obtaining circuitry 141 to be an area on which a detection process is to be performed. The detecting circuitry 143 then binarizes pixels contained in the area on which the detection process is to be performed by using a value obtained by multiplying the value in the z-axis direction of the joint 2l by 1.2 as a threshold. In the example illustrated in FIG. 7, the detecting circuitry 143 binarizes the pixels in such a manner that pixels with values equal to or larger than the threshold (pixels in the detection space 6a where the subject is not present) are turned black and that pixels with values smaller than the threshold (pixels in the detection space 6a where the subject is present) are turned white. As a result, the detecting circuitry 143 detects an area 7a in which the left hand of the person is present in the depth image. Note that the area in the depth image other than the detection space 6a is not an area on which the detection process is to be performed, and is thus shaded.

The calculating circuitry 144 calculates angle information indicating motion in the rotating direction of a part detected from the depth image information by using the coordinate information of the part. For example, the calculating circuitry 144 sets an area surrounded by the range in the x-axis direction and the range in the y-axis direction of the detection space 6a in the depth image binarized by the detecting circuitry 143 to be an area on which a calculation process is to be performed. The calculating circuitry 144 then calculates the center of gravity of the part detected by the detecting circuitry 143 in the area on which the calculation process is to be performed. The calculating circuitry 144 then calculates the angle of a long axis (principal axis of inertia) of the detected part to the horizontal direction by using the calculated center of gravity. The calculating circuitry 144 then stores the calculated angle in the angle information storage circuitry 134.

FIG. 8 is a diagram for explaining processing performed by the calculating circuitry 144 according to the first embodiment. FIG. 8 illustrates a case in which the calculating circuitry 144 calculates the center of gravity 8a of the area 7a detected in FIG. 7 and the angle of the long axis 8b.

As illustrated in FIG. 8, the calculating circuitry 144 calculates the center of gravity 8a of the area 7a by using expressions (1) and (2) below. In the expressions (1) and (2), Xc represents the X coordinate value of the center of gravity 8a, and Yc represents the Y coordinate value of the center of gravity 8a. In addition, X represents the X coordinate value of each pixel contained in the detection space 6a, and Y represents the Y coordinate value of each pixel contained in the detection space 6a. In addition, f(X, Y) is “1” if the pixel with the coordinates (X, Y) is white or “0” if the pixel is black.

Xc=ΣX×f(X,Y)/sum(f(X,Y)) (1)

Yc=ΣY x f(X,Y)/sum(f(X,Y)) (2)

The angle of the long axis 8b in the area 7a is then calculated by using expressions (3) to (6) below. In the expressions (3) to (6), σX represents a variance of pixels in the X-axis direction, and σY represents a variance of pixels in the Y-axis direction. In addition, αXY represents a covariance of X and Y, and θ represents the angle of the long axis 8b to the lateral direction (horizontal direction) of FIG. 8.

σX=Σ((X−Xc)²×f(X,Y)) (3)

σY=Σ((Y−Yc)²×f(X,Y)) (4)

σXY=Σ((X−Xc)×(Y−Yc)×f(X,Y)) (5)

θ=a tan 2(σXY,(σX−σY)) (6)

Note that the angle θ calculated here is an acute angle to the horizontal direction. The calculating circuitry 144 thus calculates the rotation angle in the rotating motion by tracking the calculated angle. In a specific example, for evaluating the rotating motion of the left forearm, the calculating circuitry 144 sets the position where the left thumb points up to 0 degrees, and expresses the supination by a positive angle and the pronation by a negative angle. In this case, the calculating circuitry 144 calculates the angles from a state in which the subject carrying out rehab holds his/her left hand at the position of 0 degrees, and tracks the calculated angles. When the subject has carried out supination, the angle changes from 0 degrees to the positive direction, and the calculating circuitry 144 thus calculates the rotation angles of 0 degrees, 45 degrees, 90 degrees, 135 degrees, . . . with the motion of supination. When the subject has carried out pronation, the angle changes from 0 degrees to the negative direction, and the calculating circuitry 144 thus calculates the rotation angles of 0 degrees, −45 degrees, −90 degrees, −135 degrees, . . . with the motion of pronation. The rotation angles of pronation may be expressed as −45 degrees, −90 degrees, −135 degrees, . . . or may be expressed as 45-degree pronation, 90-degree pronation, 135-degree pronation, . . . . If a normal range of motion of a rotating motion is assumed to be 0 to 90 degrees, for example, the calculated rotation angles are evaluated within the range of 0 to 90 degrees.

In this manner, the calculating circuitry 144 calculates the angle θ of the long axis 8b extending from the center of gravity 8a each time the area 7a is detected. The calculating circuitry 144 then tracks the calculated angle to calculate the rotation angle of the rotating motion in each frame. The calculating circuitry 144 then stores the calculated rotation angles of each frame in the angle information storage circuitry 134. Although a case in which the rotation angles of the rotating motion are stored in the angle information storage circuitry 134 has been described herein, but the embodiment is not limited thereto. For example, the calculating circuitry 144 may store the calculated angles θ in the calculating circuitry 144 itself, or may calculate and store values of angles processed depending on the type of rehab carried out by the subject.

The display controlling circuitry 145 displays motion in the rotating direction of a part. For example, the display controlling circuitry 145 displays at least one of the color image information stored in the color image information storage circuitry 133, the detection space 6a set by the setting circuitry 142, the area 7a detected by the detecting circuitry 143, the center of gravity 8a calculated by the calculating circuitry 144, and the long axis 8b calculated by the calculating circuitry 144 on the output circuitry 110.

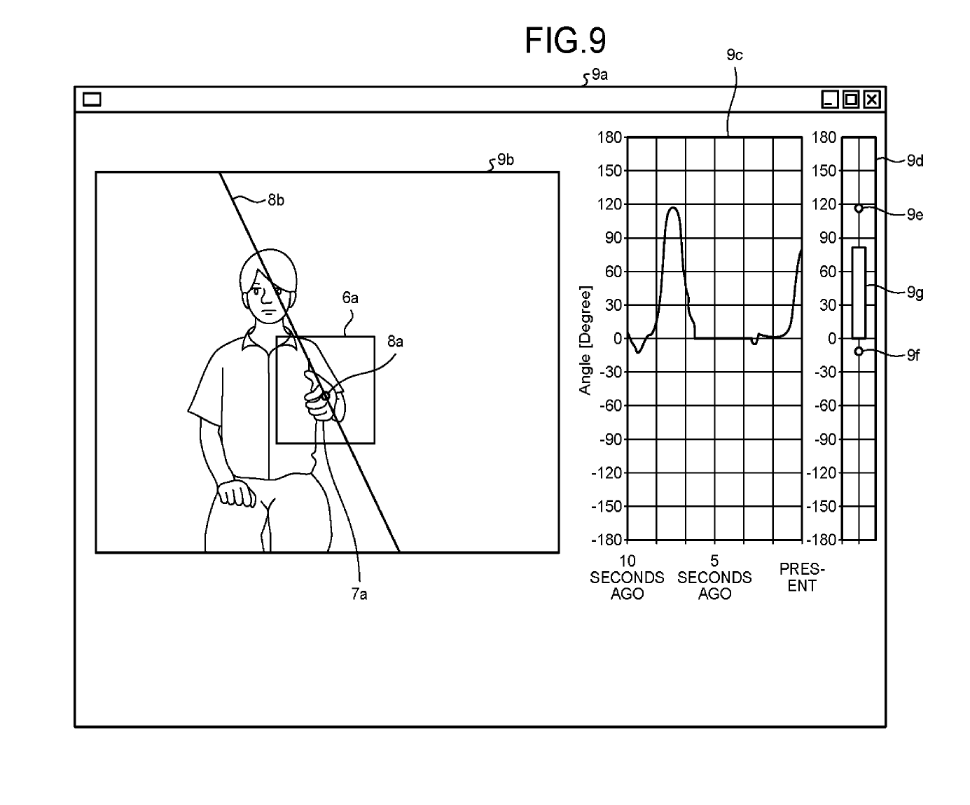

FIG. 9 is a diagram for explaining processing performed by the display controlling circuitry 145 according to the first embodiment. FIG. 9 illustrates an example of a display screen 9a displayed by the display controlling circuitry 145. The display screen 9a contains a display image 9b, a graph 9c, and a graph 9d. The display image 9b is obtained by superimposing the detection space 6a, the area 7a, the center of gravity 8a, and the long axis 8b on the color image information obtained by the obtaining circuitry 141. The graph 9c shows the rotation angle on the vertical axis and the change with time on the horizontal axis. The graph 9d shows the maximum rotation angle in the rehab being carried out, in which a point 9e represents the maximum rotation angle of supination (the minimum rotation angle of pronation), a point 9f represents the minimum rotation angle of supination (the maximum rotation angle of pronation), and a bar 9g represents the current rotation angle.

As illustrated in FIG. 9, the display controlling circuitry 145 superimposes the detection space 6a set by the setting circuitry 142, the area 7a detected by the detecting circuitry 143, and the center of gravity 8a and the long axis 8b calculated by the calculating circuitry 144 on the color image information stored in the color image information storage circuitry 133 to generate the display image 9b. The display controlling circuitry 145 displays the generated display image 9b on the output circuitry 110. Although FIG. 9 is illustrated in monochrome for the purpose of illustration, the features superimposed here are preferably displayed in different colors. For example, the detection space 6a may be displayed as a blue frame, the area 7a may be displayed as a white fill, the center of gravity 8a may be displayed as a light blue dot, and the long axis 8b may be displayed as a violet line. Alternatively, the colors are not limited to those mentioned above, but any colors that are not contained in the color image that is a background image may be selected for display. Furthermore these are not limited to the illustrated example, and the long axis 8b may be expressed by a line shorter than that in the illustrated example or by a broken line, for example. Furthermore, the long axis 8b is not limited to a line, but dots positioned on the long axis 8b may be displayed. For example, only one dot positioned on the long axis 8b may be displayed, and the motion in the rotating direction may be evaluated by using relative positions of this dot and the center of gravity.

The display controlling circuitry 145 also obtains the rotation angle in each frame from the angle information storage circuitry 134. The display controlling circuitry 145 then calculates an average value of the rotation angles of every predetermined number of frames, and plots the calculated average values on the graph 9c. The display controlling circuitry 145 updates the graph 9c each time an average value is plotted. Although FIG. 9 is illustrated in monochrome for the purpose of illustration, the plotting result (the waveform in FIG. 9) is preferably displayed as a light blue curve. Alternatively, the color is not limited to that mentioned above, but any color that is different from the scale lines may be selected for display. Furthermore, the plotted values need not necessarily be the average values, but the rotation angle of every several frames may be plotted. What is aimed at here is to continuously display the plotted graph.

The display controlling circuitry 145 also displays the point 9e and the point 9f representing the maximum rotation angles. Specifically, the display controlling circuitry 145 obtains the rotation angle in each frame from the angle information storage circuitry 134. The display controlling circuitry 145 then calculates an average value of the rotation angles of every predetermined number of frames, and stores the calculated average values. The display controlling circuitry 145 then obtains the largest value of the calculated average values of the rotation angles as the maximum rotation angle of supination and plots the obtained value as the point 9e. The display controlling circuitry 145 also obtains the smallest value of the calculated average values of the rotation angles as the minimum rotation angle of supination (the maximum rotation angle of pronation) and plots the obtained value as the point 9f. The display controlling circuitry 145 then updates and displays the graph 9d with the point 9e and the point 9f representing the maximum rotation angles and further with the bar 9g representing the current value in comparison to the points 9e and 9f. Although FIG. 9 is illustrated in monochrome for the purpose of illustration, the points 9e and 9f and the bar 9g are preferably displayed in colors different from one another. For example, the points 9e and 9f may be displayed in yellow and the bar 9g in blue. Alternatively, the color is not limited to that mentioned above, but any color that is different from the scale lines may be selected for display.

Alternatively, the display controlling circuitry 145 may display the points 9e and 9f representing the maximum rotation angles by obtaining the maximum value and the minimum value. For example, the display controlling circuitry 145 calculates the maximum value and the minimum value of the rotation angle. In a specific example, the display controlling circuitry 145 calculates a differential value of a value in the graph 9c. The display controlling circuitry 145 then obtains the value of a point where the calculated differential value has changed from a positive value to a negative value as the maximum value, and the value of a point where the differential value has changed from a negative value to a positive value as the minimum value. The display controlling circuitry 145 then plots the obtained maximum value as the maximum rotation angle of supination on the point 9e. If the point 9e is already plotted as the maximum rotation angle, the display controlling circuitry 145 compares the obtained maximum value with the value of the point 9e, and if the obtained maximum value is larger, updates the position of the point 9e with the obtained maximum value as a new maximum rotation angle. The display controlling circuitry 145 also plots the obtained minimum value as the maximum rotation angle of pronation on the point 9f. If the point 9f is already plotted as the maximum rotation angle, the display controlling circuitry 145 compares the obtained minimum value with the value of the point 9f, and if the obtained minimum value is smaller, updates the position of the point 9f with the obtained minimum value as a new maximum rotation angle. The display controlling circuitry 145 then displays the graph 9d with the point 9e and the point 9f representing the maximum rotation angles and further with the bar 9g representing the current value in comparison to the points 9e and 9f.

Although not illustrated, the display controlling circuitry 145 may display the display screen 9a in a display format different from that described above. For example, the display controlling circuitry 145 may display only rotation angles of a predetermined value or larger on the graph 9c. Alternatively, for example, the display controlling circuitry 145 may calculate a change rate of the rotation angle, the differential value of the change rate, and the like, and plot only values at several seconds before and after the time points of positive/negative inversion of the calculated values. In this manner, the display controlling circuitry 145 can create and display the graph 9c by limiting the values to be plotted to narrow down points to be focused in rehab. Furthermore, the points to be focused on in rehab may be highlighted.

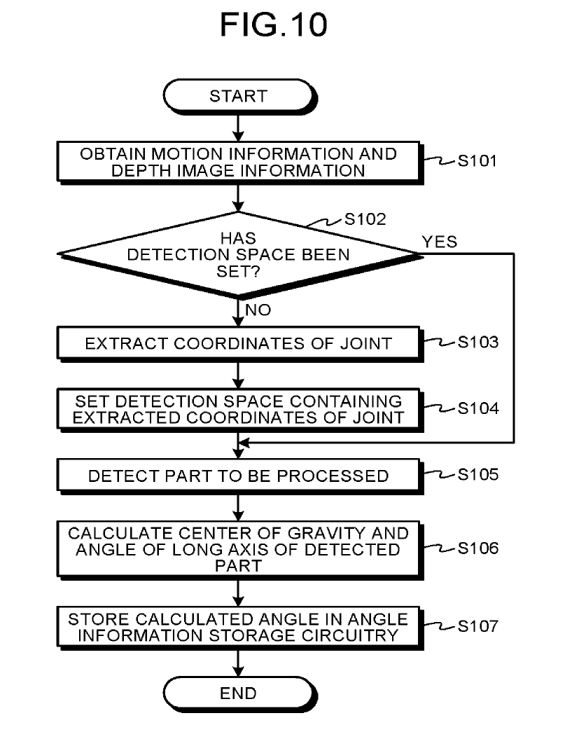

Next, procedures of processing of the motion information processing apparatus 100 according to the first embodiment will be described with reference to FIGS. 10 to 13. FIG. 10 is a flowchart for explaining an example of procedures of a calculation process according to the first embodiment.

As illustrated in FIG. 10, the obtaining circuitry 141 obtains motion information and depth image information for each frame (step S101). Subsequently, the setting circuitry 142 determines whether or not a detection space has been set (step S102). If the detection space has been set (Yes in step S102), the setting circuitry 142 proceeds to processing in step S105 without performing any process.

If the detection space has not been set (No in step S102), the setting circuitry 142 extracts coordinates of a joint to be processed from the motion information obtained by the obtaining circuitry 141 (step S103). The setting circuitry 142 then sets a detection space containing the extracted coordinates of the joint (step S104).

Subsequently, the detecting circuitry 143 binarizes the depth image information by using the detection space set by the setting circuitry 142 to detect a part to be processed (step S105).

Subsequently, the calculating circuitry 144 calculates the center of gravity and the angle of the long axis of the part detected by the detecting circuitry 143 (step S106). The calculating circuitry 144 then stores the calculated angle in the angle information storage circuitry 134 (step S107), and terminates the process.

In this manner, each time the motion information collecting circuitry 10 and the motion information processing apparatus 100 are powered on and motion information and depth image information are output from the motion information collecting circuitry 10 to the motion information processing apparatus 100, the motion information processing apparatus 100 obtains the motion information and the depth image information. The motion information processing apparatus 100 then repeats the processing from step S101 to step S107 described above using the obtained motion information and depth image information to calculate the center of gravity and the angle of the long axis of the part to be processed in real time.

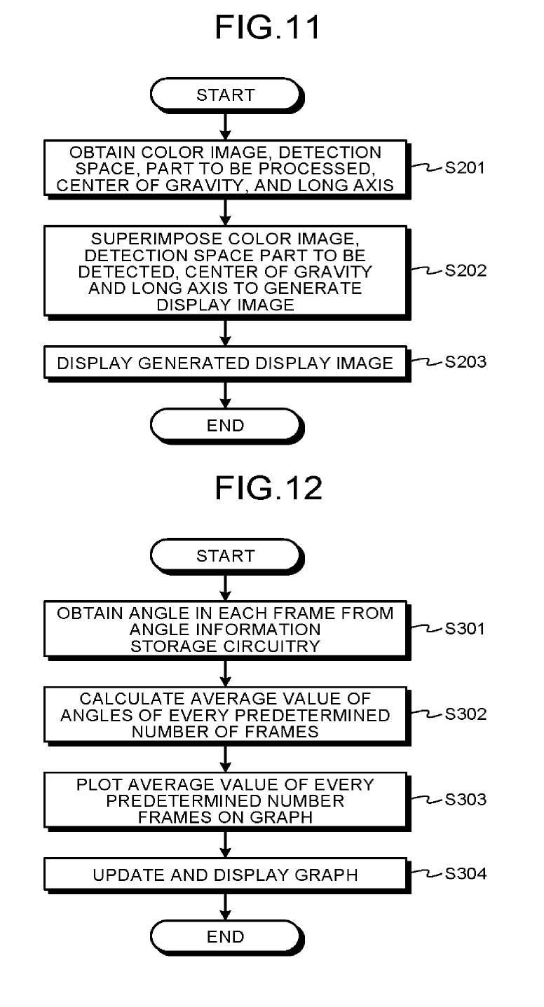

FIG. 11 is a flowchart for explaining an example of procedures of a process for displaying a display image according to the first embodiment.

As illustrated in FIG. 11, the display controlling circuitry 145 obtains information indicating a color image stored in the color image information storage circuitry 133, the detection space 6a set by the setting circuitry 142, an area 7a detected by the detecting circuitry 143, and a center of gravity 8a and the long axis 8b calculated by the calculating circuitry 144 (step S201). The display controlling circuitry 145 then superimposes the color image, the detection space 6a, the area 7a, the center of gravity 8a, and the long axis 8b to generate the display image 9b (step S202). The display controlling circuitry 145 then displays the generated display image 9b on the output circuitry 110 (step S203), and terminates the process.

In this manner, each time the motion information collecting circuitry 10 and the motion information processing apparatus 100 are powered on and color image information is stored in the color image information storage circuitry 133, the display controlling circuitry 145 repeats the processing from step S201 to step S203 described above. As a result, the display controlling circuitry 145 displays the display image 9b illustrated in FIG. 9 as a moving image substantially in real time, for example. Specifically, when a subject carrying out rehab performs rotating motion of the left arm, the display controlling circuitry 145 display a color image for allowing the subject to view the rehab carried out by the subject and also displays the detection space 6a in which the left hand is detected and the area 7a of the detected left hand. The display controlling circuitry 145 further displays the motion in the rotating direction of the left hand rotating with the rotating motion of the left arm by the direction of the long axis 8b.

FIG. 12 is a flowchart for explaining an example of procedures of a process for displaying a graph according to the first embodiment.

As illustrated in FIG. 12, the display controlling circuitry 145 obtains the rotation angle in each frame from the angle information storage circuitry 134 (step S301). Subsequently, the display controlling circuitry 145 calculates an average value of the angles of every predetermined number of frames (step S302). The display controlling circuitry 145 then plots the average value of the predetermined number of frames on the graph (step S303). The display controlling circuitry 145 shifts the plotted graph in the time direction to update the graph and displays the updated graph (step S304).

In this manner, each time a rotation angle in each frame is stored in the angle information storage circuitry 134, the display controlling circuitry 145 obtains the rotation angle and repeats the processing from step S301 to step S304 described above. As a result, the display controlling circuitry 145 displays the graph 9c illustrated in FIG. 9 substantially in real time, for example.

Note that the display by the display controlling circuitry 145 is not limited to the example described above. For example, the display controlling circuitry 145 may display a line indicating the position where the rotation angle to be evaluated is 0 degrees as a reference axis on the display image 9b. Specifically, when the position where the left thumb points up (vertical direction) is set as a reference axis (reference position) for rotation motion of the left hand, the display controlling circuitry 145 may display a line extending in the vertical direction passing through the center of gravity 8a on the display image 9b. Furthermore, if the reference axis matches with the long axis 8b, the display controlling circuitry 145 may display the matching as text information or may highlight the reference axis, for example. Furthermore, the display controlling circuitry 145 may detect an amount relating to a change in the position of the reference axis of a subject of evaluation, and display information on the detected amount relating to the change in the position. Specifically, the display controlling circuitry 145 may detect an amount by which the reference axis is shifted per unit time and display the detected amount.

Alternatively, if matter to be noted (suggestions) are set for each exercise, for example, the display controlling circuitry 145 may display these suggestions. Specifically, for a rotating motion, such information as follows may be set as suggestions: “Bend the elbow at 90 degrees so that the shoulder will not rotate together. The position at 0 degrees is the middle position of the forearm. Supination is a state in which the palm faces the ceiling. Pronation is a state in which the palm faces the floor.” In this case, the display controlling circuitry 145 may obtain the set suggestions and display the obtained suggestions on the display image 9b, for example. Furthermore, if a normal range of motion is set for each exercise, the display controlling circuitry 145 may display the normal range of motion. For example, if a normal range of motion is set to 0 to 90 degrees, the display controlling circuitry 145 may display lines indicating 0 degrees and 90 degrees, or display an area representing motion defined by these lines in a color different from the other area. Furthermore, if the rotating motion of a subject does not satisfy a normal range of motion, the display controlling circuitry 145 may output an alarm indicating abnormality, display support information to support the subject as text information or sound.

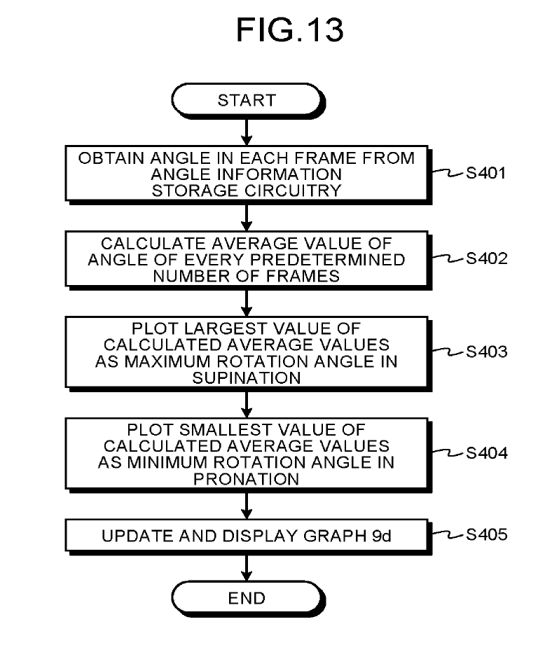

FIG. 13 is a flowchart for explaining an example of procedures of a process for displaying a maximum rotation angle according to the first embodiment.

As illustrated in FIG. 13, the display controlling circuitry 145 obtains the rotation angle in each frame from the angle information storage circuitry 134 (step S401). Subsequently, the display controlling circuitry 145 calculates an average value of the angles of every predetermined number of frames (step S402). The display controlling circuitry 145 then obtains the largest value of the average values of the rotation angles each calculated for every predetermined number of frames as the maximum rotation angle of supination and plots the obtained value as the point 9e (step S403). The display controlling circuitry 145 then obtains the smallest value of the average values of the rotation angles each calculated for every predetermined number of frames as the minimum rotation angle of supination and plots the obtained value as the point 9f (step S404). The display controlling circuitry 145 then updates and displays the graph 9d with the point 9e and the point 9f representing the maximum rotation angles and further with the bar 9g representing the current value in comparison to the points 9e and 9f (step S405).

In this manner, each time a rotation angle in each frame is stored in the angle information storage circuitry 134, the display controlling circuitry 145 obtains the rotation angle and repeats the processing from step S401 to step S405 described above. As a result, the display controlling circuitry 145 displays the graph 9d illustrated in FIG. 9 substantially in real time, for example.

Note that the procedures of processing described above need not necessarily be performed in the order described above. For example, the processing of step S403 that is a process of plotting the maximum rotation angle of supination may be performed after the processing of step S404 that is a process of plotting the minimum rotation angle of supination.

As described above, the motion information processing apparatus 100 according to the first embodiment obtains depth image information containing coordinate information and depth information of a subject present in a space in which rehabilitation is carried out. The motion information processing apparatus 100 then detects a part of the subject from the depth image information on the basis of the depth information. The motion information processing apparatus 100 then calculates angle information indicating motion in the rotating direction of the part detected from the depth image information by using the coordinate information of the part. Thus, the motion information processing apparatus 100 can evaluate the motion in the rotating direction. For example, the motion information processing apparatus 100 can evaluate motion in a rotating direction such as rotating motion of a forearm that cannot be evaluated only on the basis of coordinates of joints as described above. Specifically, the motion information processing apparatus 100 can evaluate motion in a rotating direction, which is difficult to recognize as a change in the coordinates of joints, by analyzing an image taken in a photographing direction that is substantially the same as the rotation axis.

Furthermore, for example, the motion information processing apparatus 100 sets a detection space containing the position of a joint to be processed. Thus, even when a subject is carrying out rehab at a position where the subject likes to carry out the rehab, the motion information processing apparatus 100 can automatically recognize a joint subjected to the rehab and evaluate the motion of the joint.

Furthermore, for example, the motion information processing apparatus 100 superimposes a detection space on a color image. Thus, the motion information processing apparatus 100 can make a subject recognize where to carry out rehab so that the rehab will be evaluated.

Furthermore, for example, when a subject places a part (the left hand, for example) to carry out rehab in the detected space superimposed on the color image, the motion information processing apparatus 100 detects the part and displays the detected part in a color different from those of the background image. Thus, the motion information processing apparatus 100 can make a subject recognize the part detected as a part to be evaluated in rehab.

Furthermore, for example, the motion information processing apparatus 100 superimposes a part to be processed on a color image. Thus, the motion information processing apparatus 100 can make a subject recognize the part detected as a part to be evaluated in rehab.

Furthermore, for example, the motion information processing apparatus 100 superimposes the center of gravity and the long axis of a part to be processed on a color image. Thus the motion information processing apparatus 100 can make a viewer of a display image intuitively recognize the evaluation of rehab.

### Second Embodiment

While a case in which the motion information processing apparatus 100 detects the position of a joint to be processed and sets a detection space on the basis of the detected position has been described in the first embodiment above, the embodiment is not limited thereto. For example, the motion information processing apparatus 100 may set a detection space in advance and detect a part present in the set detection space as a part to be processed. Thus, in a second embodiment, a case in which the motion information processing apparatus 100 sets a detection space in advance will be described.

A motion information processing apparatus 100 according to the second embodiment has a configuration similar to that of the motion information processing apparatus 100 illustrated in FIG. 5, but differs therefrom in part of the processing performed by the detecting circuitry 143. In the second embodiment, the description will be focused mainly on the difference from the first embodiment, and components having the same functions as those described in the first embodiment will be designated by the same reference numerals as those in FIG. 5 and the description thereof will not be repeated. Note that the motion information processing apparatus 100 according to the second embodiment need not include the motion information storage circuitry 131. Furthermore, in the motion information processing apparatus 100 according to the second embodiment, the obtaining circuitry 141 need not obtain motion information.

For example, the detecting circuitry 143 detects a part to be processed by binarizing depth image information obtained by the obtaining circuitry 141 by using the preset detection space.

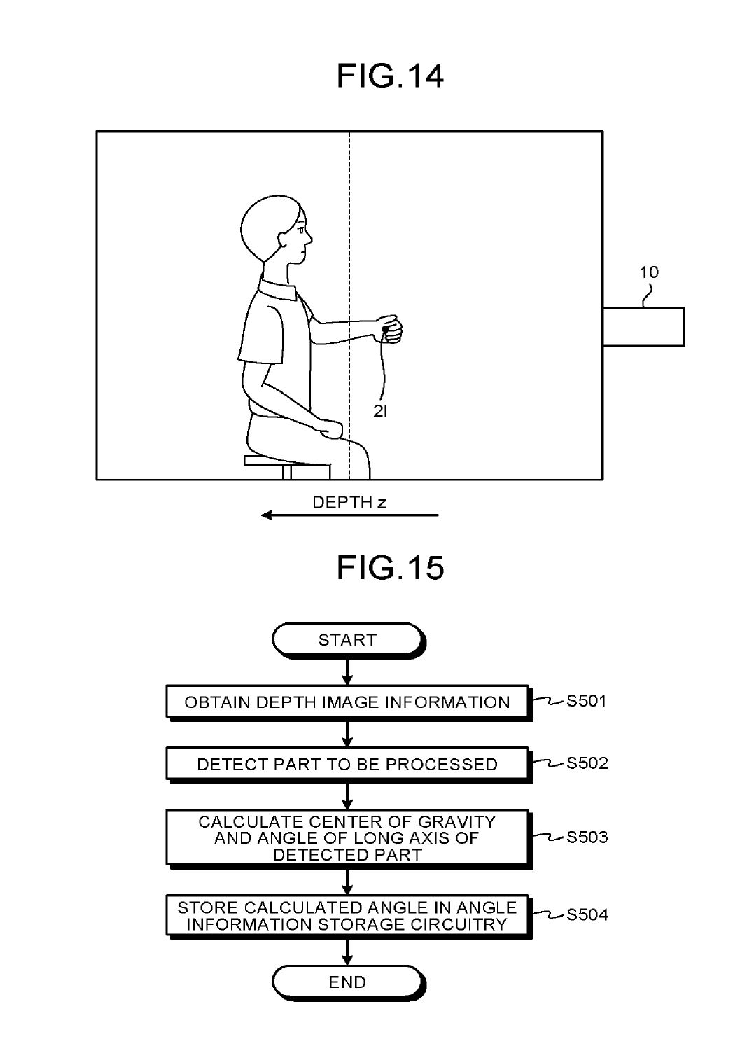

FIG. 14 is a diagram for explaining processing performed by the detecting circuitry 143 according to the second embodiment. FIG. 14 is a lateral view of a person performing rotating motion, and the leftward direction of FIG. 14 corresponds to the z-axis direction in the world coordinate system, that is, the depth. Furthermore, in FIG. 14, a space from the motion information collecting circuitry 10 to the position of a broken line is preset as a detection space from which a part to be processed is detected.

As illustrated in FIG. 14, the detecting circuitry 143 binarizes the depth image information obtained by the obtaining circuitry 141 by using the position of the broken line as a threshold. In the example illustrated in FIG. 14, the detecting circuitry 143 binarizes the pixels in such a manner that pixels with values equal to or larger than the threshold (pixels at positions farther than the broken line as viewed from the motion information collecting circuitry 10) are turned black and that pixels with values smaller than the threshold (pixels at positions closer than the broken line as viewed from the motion information collecting circuitry 10) are turned white. Thus, the detecting circuitry 143 detects the left hand to be processed by expressing an area 7a in which the left hand of the person is present in the depth image in white. Note that the detection space may be expressed by a first threshold<z<a second threshold.

Next, procedures of processing of the motion information processing apparatus 100 according to the second embodiment will be described with reference to FIG. 15. FIG. 15 is a flowchart for explaining an example of procedures of a calculation process according to the second embodiment.

As illustrated in FIG. 15, the obtaining circuitry 141 obtains depth image information for each frame (step S501). Subsequently, the setting circuitry 142 binarizes the depth image information by using the detection space on the basis of the depth image information to detect a part to be processed (step S502).

Subsequently, the calculating circuitry 144 calculates the center of gravity and the angle of the long axis of the part detected by the detecting circuitry 143 (step S503). The calculating circuitry 144 then stores the calculated angle in the angle information storage circuitry 134 (step S504), and terminates the process.

As described above, the motion information processing apparatus 100 according to the second embodiment detects a part to be processed by binarizing the pixels in such a manner that pixels in the preset detection space where the subject is present are turned white and that pixels in the detection space where the subject is not present are turned black. The motion information processing apparatus 100 can therefore evaluate motion in a rotating direction with a small processing load.

### Other Embodiments

While the first and second embodiments have been described above, various different embodiments other than the first and second embodiments can be employed.

For example, although a case in which the motion information processing apparatus 100 evaluates rotating motion of a forearm has been described in the first and second embodiments, the embodiment is not limited thereto. For example, the motion information processing apparatus 100 can also evaluate a motion of kicking one's foot up from a posture of sitting on a chair as a motion in a rotating direction.

Furthermore, for example, although a process of displaying an image on the basis of an angle calculated by the calculating circuitry 144 has been described in the first and second embodiments above, this process need not necessarily performed. Specifically, the motion information processing apparatus 100 may accumulate information indicating the angles calculated by the calculating circuitry 144 in the angle information storage circuitry 134, and read and use information indicating the accumulated angle where necessary in subsequent analysis.

Furthermore, for example, although a case in which a part is detected by the detecting circuitry 143 after a detection space set by the setting circuitry 142 has been described in the first embodiment above, the embodiment is not limited thereto. For example, the motion information processing apparatus 100 may set a detection space by the setting circuitry 142 after a part is detected by the detecting circuitry 143 as described in the second embodiment. The motion information processing apparatus 100 may then calculate the center of gravity and the angle of the long axis of a part contained in the set detection space among the detected parts.

Furthermore, for example, although a case in which the angle of the long axis 8b of the area 7a is calculated has been described in the first embodiment above, the embodiment is not limited thereto. For example, the motion information processing apparatus 100 may calculate the angle of the short axis of the area 7a.

Furthermore, for example, although a case in which the rotation angle is calculated by tracking the angle has been described in the first embodiment above, the embodiment is not limited thereto. For example, the motion information processing apparatus 100 may use the position of a thumb as a flag and track the position of the thumb to calculate the rotation angle. Specifically, the motion information processing apparatus 100 may detect a feature of an image expressing the thumb from the area 7a by pattern matching or the like, and track the relation between the position of the thumb and the position of the center of gravity to calculate the rotation angle.

Furthermore, for example, a case in which motion information collected by the motion information collecting circuitry 10 is analyzed by the motion information processing apparatus 100 to support a subject has been described in the first and second embodiments above. The embodiment, however, is not limited thereto, and the processes may be performed by a service providing apparatus on a network, for example.

Furthermore, for example, the motion information processing apparatus 100 may sense a position where a person has felt something strange in motion in a rotating direction and record the detected position. In this case, in the motion information processing apparatus 100, the controlling circuitry 140 further includes sensing circuitry for sensing the position (angle) at which a person has felt something strange in motion in a rotating direction, for example. Examples of strange things felt by a person include pain, itch, and discomfort. Hereinafter, a case in which the position where a person has felt pain is sensed will be described as an example.

For example, the sensing circuitry detects a word “ouch.” Specifically, the sensing circuitry acquires a sound recognition result of each frame from the motion information collecting circuitry 10. If a sound recognition result indicating that a person performing a motion in a rotating direction has uttered the word “ouch” is acquired, the sensing circuitry then senses angle information calculated in the frame corresponding to the sensing time as the position where the person has felt pain. The sensing circuitry stores the information indicating that the person has uttered “ouch” in association with the angle information calculated in the frame corresponding to the sensing time in the angle information storage circuitry 134, for example.

Alternatively, for example, the sensing circuitry senses a facial expression of a person when the person has felt pain. Specifically, the sensing circuitry performs pattern matching on color image information by using features of images when a person has furrowed his/her brow and features of images when a person has squeezed his/her eyes. If such a feature has been sensed by pattern matching, the sensing circuitry then senses angle information calculated in a frame corresponding to the time as a position where the person has felt pain. The sensing circuitry stores the information indicating that a facial expression when the person has felt pain has been sensed in association with the angle information calculated in the frame corresponding to the sensing time in the angle information storage circuitry 134, for example.

In this manner, the sensing circuitry senses the position (angle) where a person has felt pain in a motion in a rotating direction. Note that the sensing circuitry may record the sensed position as an indicator of a maximum range of motion in a motion in a rotating direction.

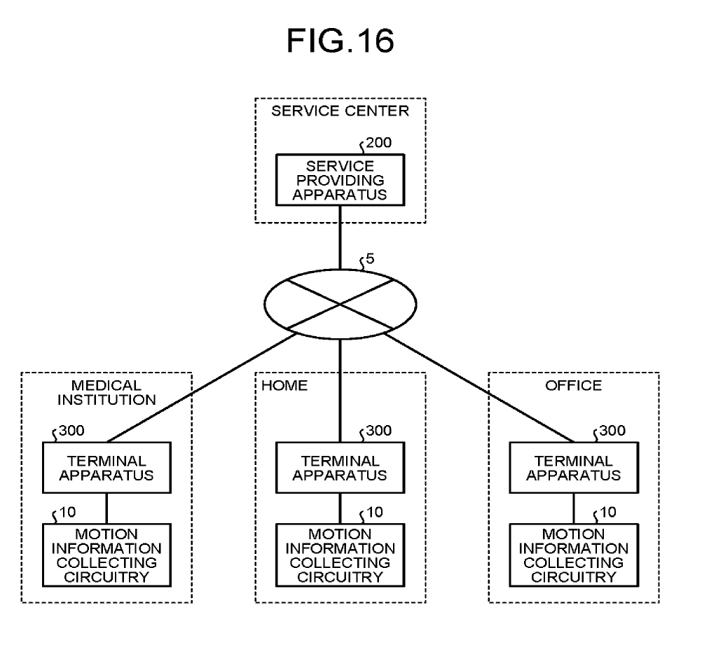

FIG. 16 is a diagram for explaining an example of application to a service providing apparatus. As illustrated in FIG. 16, a service providing apparatus 200 is installed in a service center, and connected to terminal apparatuses 300 installed in a medical institution, at home, and in an office via a network 5, for example. The terminal apparatuses 300 installed in the medical institution, at home, and in the office are each connected with a motion information collecting circuitry 10. The terminal apparatuses 300 each have a client function of using services provided by the service providing apparatus 200. For the network 5, any type of wired or wireless communication network can be used, such as the Internet and a wide area network (WAN).

The service providing apparatus 200 has functions similar to those of the motion information processing apparatus 100 described with reference to FIG. 5, and provides services to the terminal apparatuses 300 by these functions, for example. Specifically, the service providing apparatus 200 has functional units similar to the obtaining circuitry 141, the detecting circuitry 143, and the calculating circuitry 144. The functional unit similar to the obtaining circuitry 141 obtains depth information of a space in which rehabilitation is carried out. The functional unit similar to the detecting circuitry 143 detects a part contained in a detection space based on the depth information obtained by the functional unit similar to the obtaining circuitry 141 by using the depth information. The functional unit similar to the calculating circuitry 144 calculates a motion in a rotating direction of the part detected by the functional unit similar to the detecting circuitry 143. Thus, the service providing apparatus 200 can evaluate the motion in the rotating direction.

For example, the service providing apparatus 200 accepts upload of depth image information (obtained by photographing a motion in a rotating direction for a predetermined time period, for example) to be processed from a terminal apparatus 300. The service providing apparatus 200 then performs the processes described above to analyze the motion in the rotating direction. The service providing apparatus 200 allows the terminal apparatus 300 to download the analysis result.

Furthermore, the configurations of the motion information processing apparatus 100 according to the first and second embodiments are only examples, and the components thereof can be integrated or divided where appropriate. For example, the setting circuitry 142, the detecting circuitry 143, and the calculating circuitry 144 can be integrated.

Furthermore, the functions of the obtaining circuitry 141, the detecting circuitry 143, and the calculating circuitry 144 described in the first and second embodiments can be implemented by software. For example, the functions of the obtaining circuitry 141, the detecting circuitry 143, and the calculating circuitry 144 are achieved by making a computer execute motion information processing programs defining the procedures of the processes described as being performed by the obtaining circuitry 141, the detecting circuitry 143, and the calculating circuitry 144 in the embodiments described above. The motion information processing programs are stored in a hard disk, a semiconductor memory, or the like, and read and executed by a processor such as a CPU and a MPU, for example. Furthermore, the motion information processing program can be recorded distributed on a computer-readable recording medium such as a CD-ROM (Compact Disc-Read Only Memory), a MO (Magnetic Optical disk), or a DVD (Digital Versatile Disc).

Note that rehabilitation rule information, recommended status of assistance, and the like presented in the first and second embodiments described above may be those provided by various organization in addition to those provided by The Japanese Orthopaedic Association and the like. For example, various regulations and rules provided by associations as follows may be employed: “International Society of Orthopaedic Surgery and Traumatology (SICOT),” “American Academy of Orthopaedic Surgeons (AAOS),” “European Orthopaedic Research Society (EORS),” “International Society of Physical and Rehabilitation Medicine (ISPRM),” and “American Academy of Physical Medicine and Rehabilitation (AAPM&R).”

According to at least one of the embodiments described above, a motion information processing apparatus and a program therefor of the present embodiment can evaluate a motion in a rotating direction.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A motion information processing apparatus comprising:
obtaining circuitry configured to obtain depth image information containing coordinate information and depth information of a subject present in a three-dimensional space;
detecting circuitry configured to detect a part of the subject from the depth image information on the basis of the depth information; and
calculating circuitry configured to calculate angle information indicating a motion in a rotating direction of the part by using coordinate information of the part detected from the depth image information.

2. The motion information processing apparatus according to claim 1, wherein the detecting circuitry is configured to detect the part of the subject by converting the depth image information into multivalued representation on the basis of the depth information such that depth image information corresponding to a partial space of the space is extracted from the depth image information.

3. The motion information processing apparatus according to claim 2, further comprising setting circuitry configured to detect a position of a joint corresponding to the part in the space, and set a range defined by the detected position as the partial space, wherein
the obtaining circuitry is further configured to obtain skeleton information expressed by positions of joints, and
the setting circuitry is configured to extract coordinates of a joint corresponding to the part on the basis of the skeleton information and set a range defined by the extracted coordinates as the partial space.

4. The motion information processing apparatus according to claim 1, further comprising display controlling circuitry configured to display the angle information on display, wherein
the display controlling circuitry is configured to display at least one of an image in which information indicating a slope corresponding to the angle information is superimposed on the part and a graph indicating a value relating to the angle information.

5. The motion information processing apparatus according to claim 4, wherein the display controlling circuitry is further configured to display the partial space in the image.

6. The motion information processing apparatus according to claim 4, wherein the display controlling circuitry is configured to display a graph indicating a change with time in a value of the angle information.

7. The motion information processing apparatus according to claim 4, wherein the display controlling circuitry is configured to display a graph on which at least one of a maximum value and a minimum value of the angle information is plotted.

8. The motion information processing apparatus according to claim 4, wherein the display controlling circuitry is configured to detect an amount of a change in position of a reference axis of an evaluation subject and displays information on the detected amount of the change in position.

9. The motion information processing apparatus according to claim 1, further comprising sensing circuitry configured to sense a position where a person has felt pain in the motion in the rotating direction.

10. A motion information processing method comprising:
obtaining depth image information containing coordinate information and depth information of a subject present in a three-dimensional space;
detecting a part of the subject from the depth image information on the basis of the depth information; and
calculating angle information indicating a motion in a rotating direction of the part by using coordinate information of the part detected from the depth image information.
